(19) United States
(12) Patent Application Publication
(10) Pub. No.: US 2006/0018845 A1
(43) Pub. Date: Jan. 26, 2006

(54) Tooth whitening

(76) Inventors: Janette Suh Edelstein, Sudbury, MA (US); David V. Tyndall, Medway, MA (US); Ronald M. Gurge, Franklin, MA (US); Rajeev Kumar Passi, Marlborough, MA (US)

Correspondence Address: FISH & RICHARDSON P.C., P.O. BOX 1022, MINNEAPOLIS, MN, 55440-1022, US

(21) Appl. No.: 11/057,556

(22) Filed: Feb. 14, 2005

Related U.S. Application Data
(63) Continuation-in-part of application No. 10/897,637, filed on Jul. 23, 2004, now abandoned.

(51) Int. Cl. A61K 8/73; A61K 8/22

(52) U.S. Cl. 424/53

(57) ABSTRACT

Tooth whitening compositions, tooth whitening films, and methods of whitening teeth are described. The tooth whitening compositions and tooth whitening films include a tooth whitening agent and a cellulose polymer of formula (I) 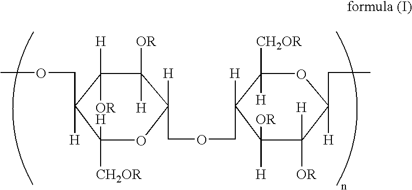
wherein; R is H, C₁₋₆ alkyl, (C₁₋₆ alkylO)ₘR², or —C(O)R¹, wherein at least one R is H, one R is C₁₋₆ alkyl or (C₁₋₆ alkylO)ₘR², and one R is —C(O)R¹; R¹ is C₅₋₁₀ aryl or C₁₋₆ alkyl, optionally substituted with at least one CO₂H; R² is H or C(O)R¹; m is 1-5; and n is 2-350. The tooth whitening compositions are may be, for example, liquids or films that are applied to a tooth for a time sufficient to whiten the tooth.

### CLAIM OF PRIORITY

This application claims priority under 35 USC §120 and is a continuation-in-part application of U.S. patent application Ser. No. 10/897,637, filed on Jul. 23, 2004, the entire contents of which are hereby incorporated by reference.

### TECHNICAL FIELD

This invention relates to whitening teeth.

### BACKGROUND

In general, it is desirable to have bright or white teeth. However, many substances that a person comes into contact with on a daily basis can stain or reduce the whiteness of one's teeth. In particular, the foods, tobacco products and fluids that one consumes tend to stain one's teeth. These products or substances tend to accumulate on the enamel layer of the tooth and form a pellicle film over the teeth. In some instances, these staining and discoloring substances can then permeate the enamel layer. The staining of teeth occurs gradually over many years, and can impart a noticeable discoloration of the enamel of a person's teeth.

A variety of over-the-counter tooth whitening systems that whiten discolored teeth are available. Some systems are applied by brushing the whitening composition on the tooth. Other systems use a tooth whitening solution disposed in a dam positioned on the tooth, and still other systems include a thin strip of plastic film applied to a surface of a user's tooth.

### SUMMARY

In general, the invention relates to tooth whitening products including a cellulose compound. The cellulose compound has the following formula (I): 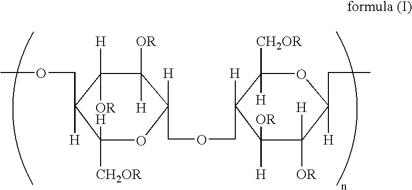
where; R is H, C₁₋₆ alkyl, (C₁₋₆ alkylO)ₘR², or —C(O)R¹, wherein at least one R is H, one R is C₁₋₆ alkyl or (C₁₋₆ alkylO)ₘR₂, and one R is —C(O)R¹; R¹ is C₅₋₁₀ aryl or C ₁₋₆ alkyl, optionally substituted with at least one CO₂H; R² is H or C(O)R₁, m is 1-5 (e.g., 1 or 2), and n is 2-350.

In one aspect, the invention features a method of whitening teeth, where the method includes applying to a tooth, for at least about 5 minutes, (for example at least about 30 minutes, about 8 hours or more), a composition including a tooth whitening agent and a compound of formula (I), for example a hydroxypropymethylcellulose ester.

In another aspect, the invention features a tooth whitening composition including; a tooth whitening agent; a volatile solvent; and a compound of formula (I). In some embodiments the composition is a liquid.

In another aspect, the invention features a product including; a tooth whitening agent, a compound of formula (I), and another component, such as a flavorant, colorant, antimicrobial agent, or other oral care active ingredient. In one embodiment, the product is a flexible film or strip including a first layer, the first layer comprising a tooth whitening agent and an adhesive; and a second layer, the second layer comprising a compound of formula (I).

In another aspect, the invention features a multi-layer oral care product including; a first tooth whitening layer positioned to contact a tooth of a user including a tooth whitening agent and an adhesive; a second, deformable backing layer including a polymer of formula (I); and a third anchor layer positioned in between the first layer and the second layer.

In general, the tooth whitening formulations described herein can be used in any of tooth whitening methods described herein.

In some embodiments, the tooth whitening composition is substantially free from metals such as copper, silver, gold, rhodium, iridium, palladium, or platinum.

In some embodiments, the invention provides a stable, whitening formulation that can remain on the surface of a tooth for a time sufficient to effect whitening of the tooth. The tooth whitening compositions can in some embodiments release the tooth whitening agent on the tooth in a manner that provides effective, targeted bleaching of the tooth.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a graph depicting the delta L* values of tooth whitening compositions at 30 minutes and 3 hours.

FIG. 2 is a graph depicting the delta b* values of tooth whitening compositions at 30 minutes and 3 hours.

FIG. 3 is a graph depicting the delta L* values of tooth whitening compositions at 30 minutes.

FIG. 4 is a graph depicting the delta b* values of tooth whitening compositions at 30 minutes.

FIG. 5 is a graph depicting delta L, delta a, and delta b values of tooth whitening films after seven days use.

### DETAILED DESCRIPTION

The tooth whitening compositions include a cellulose compound of formula (I) and a tooth whitening agent. The tooth whitening compositions can be, for example, in the form of a liquid, solid, or film.

In one embodiment in which the tooth whitening composition is a liquid, the composition includes a cellulose compound of formula (I), a tooth whitening agent, and a volatile solvent, for example, ethanol. As used herein, volatile solvent means a nonaqueous liquid with the characteristic of evaporating readily at room temperature and atmospheric pressure. The liquid composition also may include water, a pH modifier, and/or one or more of the other ingredients discussed below.

In one embodiment the tooth whitening composition is in the form of a film. The film includes a cellulose compound of formula (I), a tooth whitening agent, an adhesive, and a plasticizer. The tooth whitening film also may include one or more of the other ingredients discussed below. The film can be a single, unitary layer, or alternatively can include multiple layers, for example a dual layer film or a film containing three or more layers.

Cellulose Compounds

Examples of cellulose compounds include those cellulose compounds of formula (I) where various hydroxy moieties of the cellulose are substituted with an alkyl, a hydroxyalkyl and a carbonyl moiety, for example, to form cellulose ester derivatives. In some embodiments, the hydroxyalkyl moiety is further substituted with a carbonyl moiety to form an ester moiety.

The derivatized cellulose compounds can be tailored to exhibit desired solubility properties under the in vivo conditions present in the mouth of a user. For example, the cellulose compound can dissolve over a period of about 5 or more minutes in pH of between about 7 and about 8, for example a pH of about 7.4 at body temperature. In other instances, the cellulose compound can be formulated not to dissolve in the mouth of the user during the time of administration of the tooth whitening composition.

Examples of suitable C₁₋₆ alkyl moieties include C₁₋₆ alkyl moieties, for example, methyl, ethyl, propyl, butyl, pentyl, hexyl, etc. The C₁₋₆ alkyl moieties can be straight chain or branched alkyl. In some embodiments, the alkyl moiety is a C₁₋₃ alkyl. The compound may, for example, include between about 15% and about 30% by weight of alkyl moieties.

Examples of C₁₋₆ hydroxyalkyl moieties include, for example, hydroxymethyl, hydroxyethyl, hydroxypropyl, hydroxybutyl, hydroxypentyl, hydroxyhexyl, etc. The C₁₋₆ alkyl moieties can be straight chain branched alkyl. In some preferred embodiments, the hydroxyalkyl moiety is a C₂₋₄ hydroxyalkyl moiety such as a branched, isopropyl moiety. The compound may include, for example, between about 3% and about 12% by weight of hydroxyalkyl moiety.

Examples of carbonyl moieties include —C(O)alkyl and —C(O)aryl moieties. In some embodiments, the carbonyl moieties are further substituted, for example, with a carboxylic acid moiety. In some preferred embodiments the carbonyl moieties together with the hydroxy group of the cellulose or a C₁₋₆ hydroxyalkyl moiety form a succinate, phthalate, or acetate moiety. In some more preferred embodiments, the resulting ester is a phthalate. The compound may include, for example, between about 15% and about 40% by weight of ester moiety, preferrably between about 24% and about 31 percent of ester moiety, for example a phthalate ester.

In some preferred embodiments, the cellulose compound is an ester derivative of a hydroxypropylmethylcellulose. Preferred esters include, phthalate, succinate, and acetate as described above.

In some embodiments the cellulose compound is a block co-polymer of two different cellulose derivatives. In some preferred embodiments the block co-polymer is a copolymer of two different hydroxypropylmethylcellulose ester derivatives, for example a hydroxypropylmethylcellulose phthalate and a hydroxypropylmethylcellulose succinate.

The cellulose compound may have a molecular weight of, for example, between about 30,000 Daltons and about 150,000 Daltons, for example about 60,000 Daltons. The cellulose compound is generally soluble at pH 5 and greater. Under physiological conditions, for example pH 7.4 at 37° C., the compound generally dissolves over a period of about 5 minutes or longer. See, for example, cellulose compounds supplied by ShinEtsu of Japan.

Although the amount of cellulose compound may vary depending on the formulation of the tooth whitening composition, for example whether the composition is a liquid or a film, in general, the cellulose compound may include between about 1% and 85% by weight of the tooth composition.

Tooth Whitening Agents

Tooth whitening agents include, for example, peroxide containing compounds or any source of peroxide. Examples of whitening agents can include peroxides, metal chlorites, perborates, percarbonates, peroxyacids, persulfates, carbamides and combinations thereof. Peroxide compounds include, for example, hydrogen peroxide, urea peroxide, calcium peroxide, and mixtures thereof. Metal chlorites include, for example, calcium chlorite, barium chlorite, magnesium chlorite, lithium chlorite, sodium chlorite, and potassium chlorite. In certain preferred embodiments the whitening agent includes hydrogen peroxide or sodium percarbonate.

The whitening agent may include, for example, between about 3% and 35% by weight of the tooth whitening composition. In embodiments where the tooth whitening composition is in the form of a liquid, the whitening agent may include, for example, between about 3% and about 35% by weight of the composition. In embodiments where the tooth whitening composition is in the form of a film, the whitening agent may include, for example, between about 3% and about 35% by weight of the composition.

Adhesives

In some embodiments, the tooth whitening agent includes an adhesive, for example to help the tooth whitening composition maintain contact with a tooth. In general, any adhesive that is FDA approved for oral applications that sticks to an enamel surface when in contact with a moist tooth surface can be used. Examples of compound adhesives include hydroxyethyl cellulose, hydroxypropyl cellulose, hydroxypropylmethyl cellulose, hydroxyethylmethyl cellulose, sodium carboxymethyl cellulose, polyvinyl pyrrolidone and copolymers thereof, polyvinyl alcohol, polyethylene glycol, polyacrylic acid, polyethylene oxide, alone or in combination thereof. In some instances the adhesive is a complexed polyvinylpyrrollidone-hydrogen peroxide polymer. Other examples of adhesives include natural gums, acacia, pectin, pullulan, and synthetic polymeric adhesives such as Gantrez®, available from ISP Corp. of Wayne, N.J. In addition to the adhesive polymers described above, other examples of adhesives include medical grade silicone adhesives.

In embodiments where the tooth whitening composition is a film, the molecular weight of the adhesive polymer may generally be large enough so that an integral film can form, but not so large that immediate interfacial solubilization and adhesion to the enamel surface is impaired. In general, molecular weights range between 50,000 and 1,500,000 Daltons, and preferably between 50,000 and 500,000 Daltons.

In embodiments where the tooth whitening composition is a film, the adhesive may include between about 1% by weight and about 85% by weight of the composition. In some embodiments, for example when the film is a dual layer film, the adhesive may include between about 50% by weight and about 97% by weight of a first layer and the second layer is substantially free of the adhesive.

In embodiments where the tooth whitening composition is a liquid, the adhesive may include between about 0.1% by weight and about 50% by weight of the composition.

Plasticizers

In some embodiments the tooth whitening composition includes a plasticizer. Examples of plasticizers include glycols such as propylene glycol, polyethylene glycol, for example PEG-400, polyhydric alcohols such as glycerin and sorbitol and glycerol esters such as glycerol acetate, and citrates such as triethyl citrate. In some embodiments, a flavor oil can be a plasticizer.

In embodiments where the tooth whitening composition is a film, the plasticizer may include between about 0.1% and about 30% by weight of the film. In some preferred embodiments the plasticizer may include between about 0.5% and about 10% by weight of the composition.

In embodiments where the tooth whitening composition is a liquid, the plasticizer may include between about 0.1% and about 30% by weight of the film. In some preferred embodiments the plasticizer includes between about 0.5% and about 10% by weight of the composition.

Solvents

In some embodiments, for example when the tooth whitening composition is a liquid, the formulation includes one or more solvents. For example, in some embodiments, the tooth whitening composition includes one or more volatile solvents. Examples of volatile solvents include non-toxic alcohols such as ethanol, e.g., 200 proof ethanol, ethyl acetate, methyl ethyl ketone, and acetone. The volatile solvent may include, for example, between about 10% and about 80% by weight of the composition, preferably between about 30% and about 65%.

In some embodiments, the liquid composition also includes water. Where water is included in the composition, the water may include, for example, between about 1% and about 50% by weight of the composition, preferably between about 5% and about 30% by weight of the composition.

Other Ingredients

In some embodiments, the tooth whitening composition can include a flavoring agent. Flavoring agents include, for example, natural or synthetic essential oils as well as various flavoring aldehydes, esters, alcohols, and other materials. Examples of essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. The flavoring agent may be incorporated into the whitening composition at a concentration, for example, of between about 0.1% and about 30% by weight and preferably about 0.1% to about 20% by weight.

Coloring agents are included in some formulations of the tooth whitening compositions. For example, in some instances it is desirable to have a colored tooth whitening composition to provide a visual cue to the user that the tooth whitening composition has been applied to a tooth. As used herein, colorants include both pigments and dyes.

In some embodiments, the tooth whitening composition includes a peroxide activator. Peroxide activators may increase the bleaching efficacy of the peroxide components present in the composition. Examples of peroxide activators include metal salts such as iron, copper, or manganese, pH modifiers such as a hydroxide salt, for example sodium hydroxide, exothermic/endothermic additives such as zeolites, and photosensitizers such as titanium dioxide. Preferred pH ranges of liquid compositions are generally between about pH 3.0 and about pH 10.0, preferably between about pH 3.5 and about pH 7.5.

The tooth whitening compositions can also include oral care active ingredients such as enzymes, malodor controlling agents, cleaning agents such as phosphates, remineralizing agents, antibacterial agents, antigingivitis agents, anticarries agents, antiperiodontitis agents, tooth sensitivity agents, aphthous ulcer (canker sore) agents. Examples of agents include cetylpyridinium chloride, chlorhexidine, zinc chloride, potassium nitrate, calcium phosphate, etc.

Formulations

Liquid Compositions

In instances where the tooth whitening composition is a liquid composition, the composition generally includes between about 1% and about 50% by weight of cellulose compound, between about 3% and 35% by weight of tooth whitening agent, and between about 40% and about 90% by weight of a solvent.

The liquid composition may be applied to the tooth using a brush. In some embodiments, upon application, the cellulose compound precipitates out of the liquid composition, providing an opaque film on the tooth. This can provide a visual cue to the user that can be used to indicate that the product is both present on the tooth, and active.

In some embodiments, the liquid composition includes a multi-component mixture where each component is maintained separately prior to application of the whitening composition on the tooth. For example, in some embodiments it is desirable to prevent contact of the tooth whitening agent with another ingredient in the composition, for example a colorant, flavorant or anticarries agent, in order to prevent the peroxide from reducing the activity of the second ingredient. Accordingly, in some embodiments, the tooth whitening composition includes a two component mixture where the first component is a liquid mixture including the tooth whitening agent and the second, separate component includes one or more of a colorant, flavorant, or anticarries agent. The two components are combined upon application to the tooth.

In some embodiments, the liquid composition is left on the tooth of the user until it dissolves in the user's mouth, thus requiring no active removal step from the user. In other embodiments, after the composition has been on the tooth for a desired amount of time, the composition is removed using a brush or a rinsing agent. The composition may remain in contact with a tooth, for example, for at least about 2 minutes, for example at least about 5 minutes, at least about 10 minutes, at least about 30 minutes, at least about 1 hour, at least about 2 hours, at least about 4 hours, at least about 8 hours, or overnight.

The liquid composition can be applied, for example, directly onto the tooth without a protective shield. Alternatively, the liquid composition can be applied on the tooth using a dental tray to keep the tooth whitening composition in contact with the tooth for a desired amount of time.

The liquid composition can be made, for example, by combining the ingredients in the composition to form a stable liquid. In general, the liquid composition is prepared by first combining a solvent such as ethanol with a tooth whitening agent such as hydrogen peroxide. A cellulose compound is then added an mixed until it is dissolved. The pH is adjusted, for example with sodium hydroxide. Flavors, dyes or other ingredients are added as appropariate and q.s. with ethanol upon completion.

Film Compositions

In some embodiments, the tooth whitening composition is in the form of a film. The film may include, for example, a single, unitary layer, or alternatively can include multiple layers, for example a dual layer film or a film containing three or more layers. The film may include, for example, a cellulose compound, a tooth whitening agent, an adhesive, and a plasticizer.

The cellulose compound may include, for example, between about 1% and about 85% by weight of the film. Examples of preferred cellulose compounds include hydroxypropylmethylcellulose esters, for example phthalate, succinate, or acetate esters thereof.

The whitening agent may include, for example, between about 3% and about 35% by weight of the film. Examples of preferred whitening agents include peroxides such as hydrogen peroxide and percarbonates such as sodium percarbonate.

The adhesive may include, for example, between about 1% and about 85% by weight of the film. Examples of preferred adhesives include polyvinylpyrrolidine polymers.

The plasticizer may include, for example, between about 0.1% and about 30% by weight of the film. Examples of preferred plasticizers include polyols such as polyethylene glycol such as PEG-400.

The film may be made, for example, by making a solution of an adhesive, a cellulose compound, a plasticizer, and a tooth whitening agent dissolved in a casting solvent, for example ethanol. The solution is then poured into a case, for example a processing liner, where the casting solvent is substantially removed during the forming of the film.

In some embodiments, where the film is a unitary film, the components are evenly distributed throughout the tooth whitening strip. In other embodiments, for example where the strip is a dual layer strip the components can be separated into individual layers of the film. For example a dual layer film can include a first, tooth contacting layer that includes an adhesive and a tooth whitening agent. The second layer can include a cellulose compound that acts a barrier to keep the tooth whitening agent in contact with the tooth and prevent the tooth whitening agent from becoming washed away from the tooth in the saliva of the user before the tooth whitening agent has acted upon the tooth. In still other embodiments, the film includes three or more layers, each layer can include one or more of a tooth whitening agent, oral active ingredient, adhesive, dye, cellulose compound, etc.

In some instances, the film includes a backing layer, an oral care layer, and an anchor layer. Examples of such films are provided in U.S. Patent Publication No. 2004/0005277, the entire contents of which is hereby incorporated by reference.

The backing layer may include a thin, flexible layer of permanently deformable material. As used herein “permanently deformable” means that the backing layer retains any shape into which it is formed by application of slight pressure e.g., less than about 250,000 Pascals per square centimeter. Thus, the film can readily conform to the surface of the teeth and adjoining soft tissue across the dental arch in the user's mouth without tearing, cracking or breaking. The backing layer material generally has visco-elastic properties which allow the backing layer to creep as well as bend when pressure is applied to the device. The backing layer can include a non-polymeric material such as a wax (e.g., microcrystalline or paraffin waxes), a tackifier (e.g., a natural or synthetic resin, such as a hydrocarbon resin), a hydrocarbon resin, or mixtures thereof. In some instances, the backing layer can include a polymer, for example a compound of formula (I).

In some embodiments, the backing layer is flavored. For example, the backing layer can include a flavorant forumulated therein. The flavorant can be incorporated, for example, into a compound, which releases the flavorant into the mouth of the user. In some instances, the compound is a compound of formula (I). The formulated flavorant can be cast as a solution onto a wax in the backing layer or laminated onto the backing.

In general, the flavorant formulation includes from about 1% to about 50% by weight compound, from about 1% to about 30% by weight of flavorant, such as a flavor oil, and from about 20% to about 90% solvent, such as ethanol (e.g., 190 proof ethanol). The formulated flavor can be uniformly distributed on the backing layer, and/or can be applied in a patterned manner such as stripes or polka-dots. The intensity and amount of flavor can be adjusted, for example, by controlling the thickness of the cast film.

In some embodiments, the backing layer can include an oral care active ingredient such as an anticaries agent or an antimicrobial agent in addition to or in place of the flavorant. The oral care active ingredient can be formulated in a compound such as a compound of formula (I).

In some instances, the compound of formula (I) releases a flavorant in a controlled manner. For example, the composition and/or thickness of the compound of formula (I) can be varied to modify the manner in which the flavorant is released into the mouth of the user.

The anchor layer generally includes a thin, flexible layer of material such as open-cell foam or a non-woven material (e.g., polyester or polypropylene) which is located immediately adjacent to the backing layer. The opposing faces of the backing and anchor layer material are in contact with one another essentially along their entire surfaces, and the material comprising the backing layer penetrates slightly into spaces between the cells of the opposing side of the foam forming the anchor layer. Generally, the face of the backing layer contacting the anchor layer is softened by heating prior to laminating the backing layer to the anchor layer.

The oral care layer generally includes at least one oral care agent and at least one hydrophilic polymer, and is located immediately adjacent to the side of the anchor layer which is not attached to the backing layer. The opposing faces of the oral care and anchor layers are in contact with one another and form an adhesive bond between those layers. The oral care layer is disposed on the anchor layer and is minimally invested in the foam. As used herein, “minimally invested” means that the oral care layer fills only the surface depressions in the anchor layer foam, but does not appreciably penetrate below the surface of the anchor layer. The oral care layer is generally co-extensive with the anchor layer.

The rate at which the whitening agent is solubilized (in the case of a solid whitening agent) and subsequently released to a tooth surface can be controlled by properties such as, but not limited to, the film thickness, compound properties such as structure and molecular weight and type, properties of whitening agent and the concentration of the whitening agent.

The thicknesses of the film layer may affect the residence time of the composition. The residence time of a layer including the cellulose compound may, for example, be determined by a combination of the composition of the compound and the thickness of the film. However, inclusion of a water soluble, compound precast film as one of the layers of the device may also increase the residence time, since the overall thickness of the device is increased.

The total thickness of the film may, for example, affect the mouth feel of the product, which can relate to user acceptance and compliance. A very thick device can become more noticeable with respect to “mouth feel”, and may cause the user to discontinue its use prematurely, thus compromising efficacy.

Additionally, the thickness of the film may also affect its ability to conform and adhere to teeth in an efficient manner. Typically, thin films are preferred since they can be easily applied and bent around the teeth, minimizing the amount of unattached area in the form of edges and comers that could cause the film to be accidentally pulled off the teeth. However, if the film is too thin, it may lose tensile strength and rip during its application. In general, a preferred thickness for the film is between about the overall thickness of the device is between about 20 micrometers and about 1500 micrometers, and more preferably between about 50 micrometers and about 1000 micrometers.

The film can be applied to the tooth, for example, by pressing the film onto the tooth of the user. In some embodiments, the film will dissolve in the mouth of the user, and thus will not require a separate removal step, for example using a brush or a rinse. In other embodiments, the film will not completely dissolve in the mouth of a user and will require removal by the user, for example, by simply pulling of the remaining strip or using a brush or rinse.

The film may remain in contact with the tooth, for example, for at least about 2 minutes, for example at least about 5 minutes, at least about 10 minutes, at least about 30 minutes, at least about 1 hour, at least about 2 hours, at least about 4 hours, or overnight.

### EXAMPLES

### Example 1

### Liquid Tooth Whitening Composition

The following tooth whitening composition was prepared by combining the below ingredients together, forming a stable, liquid composition.

| Ingredient | % by weight |
|---|---|
| Ethanol | 57.14 |
| Hypromellose phthalate | 20.00 |
| Hydrogen peroxide (35%) | 22.86 |
| TOTAL | 100.00 |

The formulation was tested using a tooth whitening efficacy model, which is designed to measure the whitening effect of various whitening products, and allows for the comparison of those products. Bovine teeth are used, and the shade, i.e., the color of the teeth, is measured before and after a treatment with a whitening product using a calorimeter, such as the MHT SpectroShade System. The difference in tooth shade before and after treatment is a measure of the efficacy of the product.

The bovine teeth are prepared using a medical scalpel to remove tissue from the tooth surface. In instances where the root is removed, the tissue is scraped from the root/tooth junction to expose the root surface. If the root remains on the tooth, the flesh is removed as completely as possible.

The tooth is prophied with commercial prophy paste for about 1-2 minutes per tooth to remove all extrinsic stain. Excess paste is then removed from the tooth.

Once cleaned, the tooth is dabbed dry and painted with the tooth whitening formulation on the buccal surface of the tooth. The tooth is allowed to dry for 2 minutes and then placed for an allocated time in either water or artificial saliva. After the treatment, the tooth is wiped of excess film with a tissue and placed in deionized water for at least 30 minutes. The tooth shade is then measured in the SpectroShade, and L*, a*, and b* values are recorded.

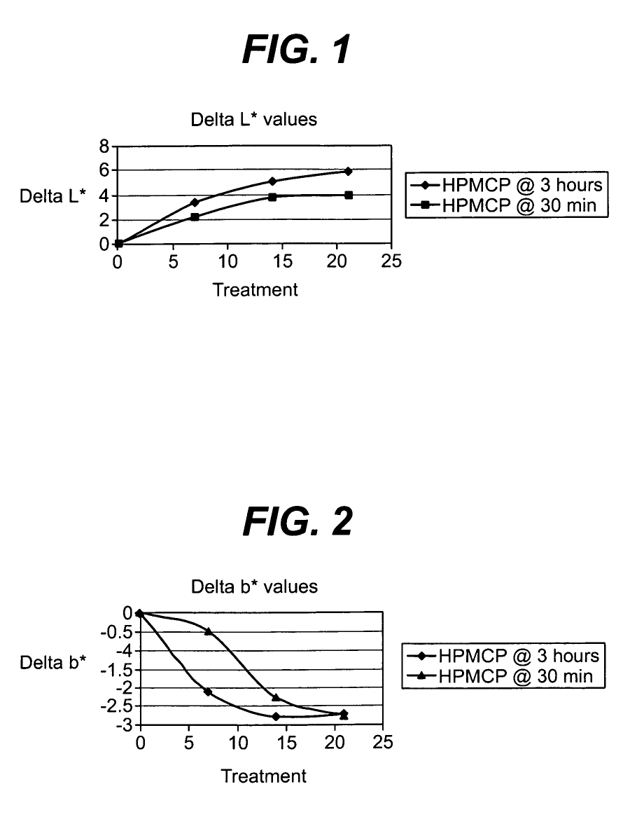

FIGS. 1 and 2 show the Delta L*, and Delta b* values respectively for the tooth whitening composition at times of 30 minutes and 3 hours. As can be seen in the figures, the tooth whitening composition performed comparably to another commercially available product, and continued to show improved whitening effects when left on the tooth for 3 hours.

### Example 2

### Liquid Tooth Whitening Compositions Using Various Grades of Hypromellose phthalate

The following formulations using three different grades of hypromellose phthalate: 50, 55, and 55S were prepared and evaluated using the methods described above in Example 1.

The three grades are distinguished in their respective phthalyl content (PC), methoxyl content (MC), hydroxypropyl content (PHC) and viscosity as shown below. The 55 and 55S grades vary by viscosity with 55S having a higher degree of polymerization.

| | PC | MC | HPC | V |
|---|---|---|---|---|
| Hypromellose phthalate | 24% | 20-24% | 6-10% | 55 cSt |
| 50 (HP-50) | | | | |
| Hypromellose phthalate | 31% | 18-22% | 5-9% | 40 cSt |
| 55 (HP-55) | | | | |
| Hypromellose phthalate | 31% | 18-22% | 5-9% | 170 cSt |
| 55S (HP-55S) | | | | |

| Ingredient | % by weight |
|---|---|
| Ethanol | 55.00 |
| Hypromellose phthalate | 20.00 |
| Hydrogen Peroxide (35%) | 22.86 |
| | (equivalent to 8.00% |
| | Hydrogen Peroxide) |
| Sodium hydroxide (50%) | <0.5 |

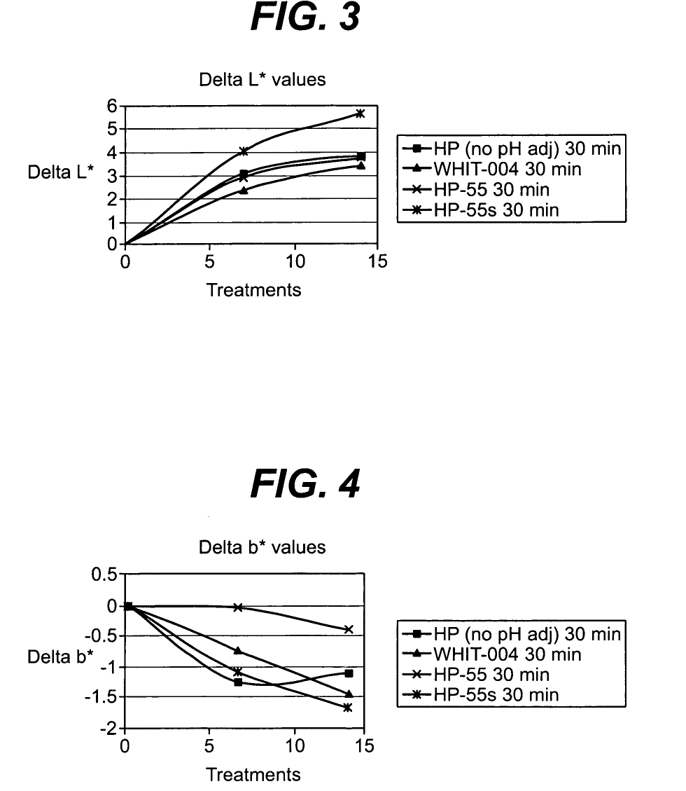

The Delta L*, and Delta b* for the various compositions are shown at 30 minutes in FIGS. 3 and 4 respectively. As can see from the figures, the tooth whitening composition having the HP-55S showed the greatest delta values in each example.

### Example 3

### Additional Liquid Tooth Whitening Compositions

Other examples of effective tooth whitening compositions are shown below.

| Basic formulation | % by weight |
|---|---|
| Ethanol, 95% | 56.89 |
| Hypromellose phthalate | 20.00 |
| Hydrogen Peroxide, 35% in water | 25.71 |
| Sodium Hydroxide, 50% in water | q.s. to pH 4-5 |
| Total | 100.00 |

Basic Flavored & Sweetened Formulation % by Weight

| | |
|---|---|
| Ethanol, 95% | 53.04 |
| Hypromellose phthalate | 20.00 |
| Hydrogen Peroxide, 35% in water | 25.71 |
| Menthol | 0.600 |
| Methyl Salicylate | 0.075 |
| Eucalyptol | 0.075 |
| Sodium Hydroxide, 50% in water | q.s. to pH 4-5 |
| Total | 100.00 |

### Example 4

### Tooth Whitening Films

Examples of tooth whitening films are provided in Table 1 below. The levels of ingredients in the final film generally determine its characteristics (i.e., film strength, film flexibility, consumer attributes such as overall mouth feel, etc.).

TABLE 1

| Ingredient | A | B | C | D | E | F | G | H | I |
|---|---|---|---|---|---|---|---|---|---|
| Ethanol | 70 | 60 | 50 | 65 | 65 | 67 | 69.5 | 60 | 63.33 |
| PVP K-90 | 20 | 20 | 20 | 11.25 | 10 | 15 | 15 | 20 | 20 |
| HPMCP 55S | | | | 3.75 | 5 | | | | |
| PEG-400 | | 10 | 20 | 10 | 10 | 10 | 10 | 10 | 10 |
| Sodium Percarbonate | 10 | 10 | 10 | 10 | 10 | 8 | 5.5 | 10 | 6.67 |
| H&R Flavor (Peroxide Stable) | | | | | | | | | |
| Total | 100 | 100 | 100 | 100 | 100 | 100 | 100 | 100 | 100 |
| Film Properties | Brittle | Flexible | Soft | Flexible | Flexible | Flexible | Flexible | Flexible | Flexible |
| Film Thickness (mm) | 0.371 | 0.373 | N/A | 0.341 | 0.330 | 0.389 | 0.372 | 0.607 | 0.600 |
| Std. Dev. (n = 16) | 0.035 | 0.035 | N/A | 0.024 | 0.030 | 0.058 | 0.044 | 0.104 | 0.037 |
| H₂O₂ (mg/cm²) | 4.46 | 3.59 | N/A | 5.11 | 4.97 | 5.76 | 3.50 | 7.45 | 3.81 |
| Std. Dev. (n = 3, 9 cm²) | 0.17 | 0.37 | N/A | 0.15 | 0.32 | 0.23 | 0.16 | 0.61 | 0.19 |
| % H₂O₂ | 9.56 | 7.40 | N/A | 8.63 | 8.46 | 7.06 | 5.47 | 7.51 | 5.67 |
| Std. Dev. (n = 3, 9 cm²) | 0.03 | 0.03 | N/A | 0.04 | 0.29 | 0.07 | 0.09 | 0.03 | 0.06 |
| % Percarbonate | 29.36 | 22.71 | N/A | 26.51 | 25.97 | 21.67 | 16.78 | 23.05 | 17.42 |
| Std. Dev. (n = 3, 9 cm²) | 0.08 | 0.08 | N/A | 0.14 | 0.88 | 0.21 | 0.28 | 0.10 | 0.17 |

In general, films obtained from casting solution A were brittle (not acceptable for a flexible strip). However, upon the addition of PEG-400 as a plasticizer (B) flexible films were obtained, which conformed and adhered to teeth. The dissolution rate of the films can be controlled by variation of film thickness or by adding specific amounts of the hydroxypropyl methylcellulose esters (D and E). These films are robust and may be utilized as a single layered strip. The peroxide level can also be controlled by different loadings of whitening active in the casting film solutions (F-I).

### Example 5

### Tooth Whitening Films with Hydrogen Peroxide Whitening Agent

The following Table 2 provides examples of tooth whitening films where hydrogen peroxide is used as the whitening agent.

TABLE 2

| Ingredient | A2 | B2 | C2 | D2 | E2 | F2 |
|---|---|---|---|---|---|---|
| Ethanol (95%) | 58.57 | 63.57 | 55.71 | 60.71 | 54.26 | 52.86 |
| PVP K-90 | 20 | 15 | 20 | 15 | 20 | 20 |
| HPMCP 55S | | | | | | |
| PEG-400 | 10 | 10 | 10 | 10 | 10 | 10 |
| H₂O₂ (35%) | 11.43 | 11.43 | 14.29 | 14.29 | 15.74 | 17.14 |
| Total | 100 | 100 | 100 | 100 | 100 | 100 |
| Film Properties | Flexible | N/A | Flexible | N/A | Flexible | Flexible |
| Film Thickness (mm) | 0.317 | N/A | 0.319 | N/A | 0.470 | 0.533 |
| Std. Dev. (n = 16) | 0.020 | N/A | 0.023 | N/A | 0.042 | 0.040 |
| H₂O₂ (mg/cm²) | 4.54 | N/A | 4.18 | N/A | 5.68 | 6.56 |
| Std. Dev. (n = 3, 9 cm²) | 0.15 | N/A | 0.62 | N/A | 1.14 | 0.83 |
| % H₂O₂ | 8.68 | N/A | 8.84 | N/A | 9.56 | 10.09 |
| Std. Dev. (n = 3, 9 cm²) | 0.50 | N/A | 0.50 | N/A | 0.32 | 0.49 |

The preferred casting solution where hydrogen peroxide is the whitening agent was found to contain 20% PVP and 10% PEG-400. The initial amounts of peroxide loading allows for easy control of the final hydrogen peroxide content of the films. These adhesive layered peroxide films are utilized in a dual-layered oral care delivery system.

### Example 6

### Multi Layer Film Compositions

Table 3 below provides examples of various compositions used in dual layer films. The films include an inner layer and an outer layer.

TABLE 3
Outer Layer Casting Formulation

| Component | A-1 | A-2 | A-3 | A-4 | A-5 | A-6 |
|---|---|---|---|---|---|---|
| Ethanol (200 proof) | 75.00 | 74.99 | 75.00 | 74.99 | 75.00 | 74.99 |
| HPMCP 55S | 15.00 | 15.00 | | | | |
| HPMCP 55 | | | 15.00 | 15.00 | | |
| HPMCP 50 | | | | | 15.00 | 15.00 |
| PEG (400 or 600) | 10.00 | 10.00 | 10.00 | 10.00 | 10.00 | 10.00 |
| FD&C Blue #1 | | 0.01 | | 0.01 | | 0.01 |
| Average | 100.00 | 100.00 | 100.00 | 100.00 | 100.00 | 100.00 |

Inner Layer Casting Formulation

| | B-1 | B-2 | B-3 | B-4 | B-5 | B-6 |
|---|---|---|---|---|---|---|
| Ethanol (200 proof) | 64.91 | 60.00 | 77.50 | 59.47 | 56.50 | 58.13 |
| Poly (Vinyl Pyrrolodone)- | 29.07 | | | | | 14.54 |
| H₂O₂ Complex | | | | | | |
| (Peroxydone K-90) | | | | | | |
| Poly (Vinyl Pyrrolodone)- | | | | | 32.09 | 20.21 |
| H₂O₂ Complex | | | | | | |
| (Peroxydone K-30) | | | | | | |
| VP/VA Copolymer | | | | 33.22 | | |
| (Copovidone)- | | | | | | |
| H₂O₂ Complex | | | | | | |
| (Plasdone S-630) | | | | | | |
| Sodium Percarbonate | | 10.00 | | | | |
| VP/VA Copolymer | | | 15.00 | | 3.91 | |
| (Copovidone | | | | | | |
| Plasdone S-630) | | | | | | |
| Poly (Vinyl | | 20.00 | | | | |
| Pyrroloidone) K-90 | | | | | | |
| PEG (400 or 600) | 6.02 | 10.00 | 7.50 | 7.31 | 7.50 | 7.12 |
| Average | 100.00 | 100.00 | 100.00 | 100.00 | 100.00 | 100.00 |

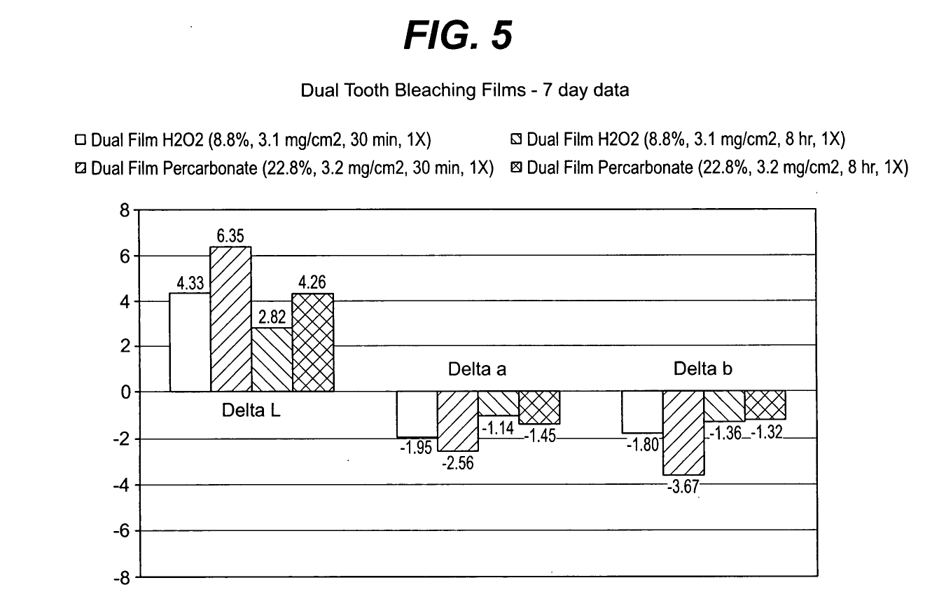

FIG. 5 shows Delta L, Delta a, and Delta b values for two different dual layer films: Film Y (a hydrogen peroxide containing film) having a combination outer layer A-2 and inner layer B-1; and Film Z (a sodium percarbonate containing film) having a combination outer layer A-2 and inner layer B-2. Upon drying, Film Y contained 8.8% hydrogen peroxide by weight and 3.1 mg/cm². Upon drying, Film Z contained 22.8 sodium percarbonate by weight and 3.2 mg/cm². Measurements of tooth whitening were taken at seven days under conditions of 30 minuted use once daily and 8 hour use once daily for both of the films.

Combinations of the inner and outer casting formulations were prepared as shown in Table 4 below. As can be seen from Table 4, each strip architecture can provide compositions that have varying residence times of the teeth of a user.

TABLE 4

| | | Strip | Residence |
|---|---|---|---|
| Outer Layer | Inner Layer | Architecture | Time on Teeth |
| A-1 or A-2 | B-1 | Dual | >6 hrs |
| A-1 or A-2 | B-2 | Dual | 5 min-4 hrs |
| A-1 or A-2 | B-3/B-2 | Triple | >6 hrs |
| A-1 or A-2 | B-4/B-2 | Triple | >6 hrs |
| A-1 or A-2 | B-5 | Dual | >6 hrs |
| A-1 or A-2 | B-6 | Dual | >6 hrs |
| A-3 or A-4 | B-1 | Dual | 5 min-4 hrs |
| A-3 or A-4 | B-2 | Dual | 5 min-4 hrs |
| A-3 or A-4 | B-3/B-2 | Triple | 5 min-4 hrs |
| A-3 or A-4 | B-4/B-2 | Triple | 5 min-4 hrs |
| A-3 or A-4 | B-5 | Dual | 5 min-4 hrs |
| A-3 or A-4 | B-6 | Dual | 5 min-4 hrs |
| A-5 or A-6 | B-1 | Dual | 5 min-2 hrs |
| A-5 or A-6 | B-2 | Dual | 5 min-2 hrs |
| A-5 or A-6 | B-3/B-2 | Triple | 5 min-2 hrs |
| A-5 or A-6 | B-4/B-2 | Triple | 5 min-2 hrs |
| A-5 or A-6 | B-5 | Dual | 5 min-2 hrs |
| A-5 or A-6 | B-6 | Dual | 5 min-2 hrs |

### Example 7

### Multi Layer Film Composition Including an Oral Care Layer an Anchor Layer, and a Backing Layer

The backing layer includes a flavorant oil formulated in an HPMCP-50 polymer, which is coated onto a wax backing. The flavorant formulation includes 190 Proof Ethanol, 59.5%; HPMCP-50 polymer, 20%, flavor oil, 20%, and sodium saccharin, 0.5%.

A number of embodiments of the invention have been described. Nevertheless, it will be understood that various modifications may be made without departing from the spirit and scope of the invention. Accordingly, other embodiments are within the scope of the following claims.

1. A method of whitening teeth, the method comprising;
applying to a tooth, for at least about 5 minutes, a composition comprising a tooth whitening agent and a compound of formula (I) 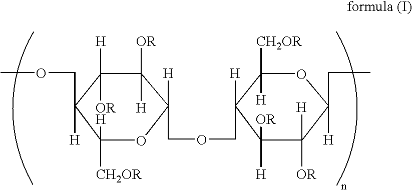
wherein;
R is H, C₁₋₆ alkyl, (C₁₋₆ alkylO)ₘR², or —C(O)R¹, wherein at least one R is H, one R is C₁₋₆ alkyl or (C₁₋₆ alkylO)ₘR², and one R is —C(O)R¹;
R¹ is C₅₋₁₀ aryl or C₁₋₆ alkyl, optionally substituted with at least one CO₂H;
R² is H or C(O)R¹;
m is 1-5; and
n is 2-350.

2. The method of claim 1, wherein m is 1.

3. The method of claim 1, wherein at least one R² is C(O)R¹ substituted with at least one —CO₂H.

4. The method of claim 1, wherein at least one R is H, at least one R is C₁₋₃ alkyl, at least one R is C₂₋₆ alkylOR², and at least one R is C(O)R₁.

5. The method of claim 1, wherein at least one R is H, at least one R is methyl, at least one R is hydroxypropyl, and at least one R is C(O)R¹; and
R¹ is C₅₋₁₀ aryl substituted with CO₂H or C₁₋₆ alkyl substituted with CO₂H.

6. The method of claim 5, wherein at least one R is H, at least one R is methyl, at least one R is hydroxypropyl, and at least one R is phthalate, wherein hydroxypropyl is optionally substituted with phthalate.

7. The method of claim 5, wherein at least one R is H, at least one R is methyl, at least one R is hydroxypropyl, and at least one R is acetate, wherein hydroxypropyl is optionally substituted with acetate.

8. The method of claim 5, wherein at least one R is H, at least one R is methyl, at least one R is hydroxypropyl, and at least one R is succinate, wherein hydroxypropyl is optionally substituted with succinate.

9. The method of claim 1, wherein the compound of formula (I) is a hydroxypropyl methylcellulose ester.

10. (canceled)

11. The method of claim 1, wherein the compound of formula (I) has a molecular weight between about 30,000 and 150,000 Daltons.

12-15. (canceled)

16. The method of claim 13, wherein the compositions comprises two liquids that are kept separate until application to the tooth.

17. The method of claim 13, wherein the composition further comprises an alcohol.

18. The method of claim 1, wherein the composition is in the form of a flexible film.

19. The method of claim 18, wherein the film comprises only one layer.

20. The method of claim 18, wherein the film comprises only two layers.

21. The method of claim 18, wherein the film comprises at least three layers.

22. The method of claim 18, wherein the film further comprises an adhesive.

23-24. (canceled)

25. The method of claim 18, wherein the film comprises a first layer and a second layer, and the first layer comprises an adhesive and a tooth whitening agent and a second layer comprises a compound of formula (I).

26-27. (canceled)

28. The method of claim 1, further comprising removing the composition from the tooth using a rinsing agent or a brush.

29. The method of claim 1, further comprising removing the composition from the tooth, wherein the composition is in an intact form.

30. The method of claim 1, wherein the composition dissolves on the tooth.

31. The method of claim 1, wherein the composition, upon application to the tooth, becomes opaque.

32. (canceled)

33. The method of claim 1, wherein the whitening agent is a peroxide.

34. (canceled)

35. A tooth whitening composition comprising;
a tooth whitening agent;
a volatile solvent; and
a compound of formula (I) 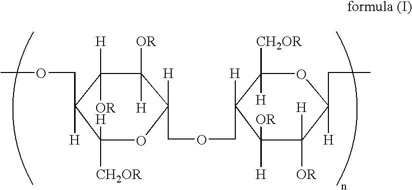
wherein;
R is H, C₁₋₆ alkyl, (C₁₋₆ alkylO)ₘR², or —C(O)R¹, wherein at least one R is H, one R is C₁₋₆ alkyl or (C₁₋₆ alkylO)ₘR², and one R is —C(O)R¹;
R¹ is C₅₋₁₀ aryl or C₁₋₆ alkyl, optionally substituted with at least one CO₂H;
R² is H or C(O)R¹;
m is 1-5; and
n is 2-350.

36. The tooth whitening composition of claim 35, wherein the solvent is ethanol.

37. The tooth whitening composition of claim 35, further comprising a plasticizer.

38. The tooth whitening composition of claim 35, further comprising an adhesive.

39. A flexible film comprising;
a first layer, the first layer comprising a tooth whitening agent and an adhesive; and
a second layer, the second layer comprising a compound of formula (I); 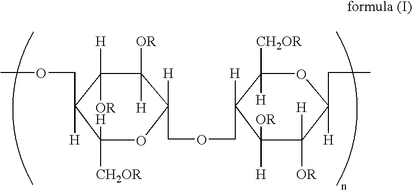
wherein;
R is H, C₁₋₆ alkyl, (C₁₋₆ alkylO)ₘR², or —C(O)R¹, wherein at least one R is H, one R is C₁₋₆ alkyl or (C₁₋₆ alkylO)ₘR², and one R is —C(O)R¹;
R¹ is C₅₋₁₀ aryl or C₁₋₆ alkyl, optionally substituted with at least one CO₂H;
R² is H or C(O)R¹;
m is 1-5; and
n is 2-350.

40. The flexible film of claim 39, wherein the compound of formula (I) is a hydroxypropylmethyl cellulose ester.

41. The flexible film of claim 39, wherein the compound of formula (I) is hydroxypropylmethyl cellulose phlalate.

42. The flexible film of claim 39, wherein the second layer further comprises a flavorant.

43. The flexible film of claim 39, wherein the second layer further comprises a colorant.

44. The flexible film of claim 39, wherein the second layer further comprises an oral care active ingredient.

45. The flexible film of claim 39, further comprising a third anchor layer disposed between the first layer and second layer.

46. The flexible film of claim 45, wherein the third anchor layer comprises an open cell foam.

47. The flexible film of claim 45, wherein the third anchor layer comprises a non-woven material.

48. (canceled)

49. The flexible film of claim 39, the first layer further comprising a hydrophilic polymer.

50. A multi-layer oral care product comprising;
a first tooth whitening layer positioned to contact a tooth of a user comprising a tooth whitening agent and an adhesive;
a second, deformable backing layer comprising a polymer of formula (I) 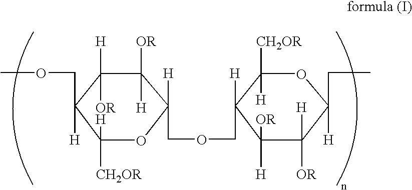
wherein;
R is H, C₁₋₆ alkyl, (C₁₋₆ alkylO)ₘR², or —C(O)R¹, wherein at least one R is H, one R is C₁₋₆ alkyl or (C₁₋₆ alkylO)ₘR², and one R is —C(O)R¹;
R¹ is C₅₋₁₀ aryl or C₁₋₆ alkyl, optionally substituted with at least one CO₂H;
R² is H or C(O)R¹;
m is 1-5; and
n is 2-350; and
a third anchor layer positioned in between the first layer and the second layer.

51-54. (canceled)

55. The multi-layer oral care device of claim 50, wherein the second layer further comprises a flavorant.

56-58. (canceled)

59. A method of whitening teeth, the method comprising, applying to a tooth, for at least about 5 minutes, a product comprising a tooth whitening agent and a compound of formula (I) 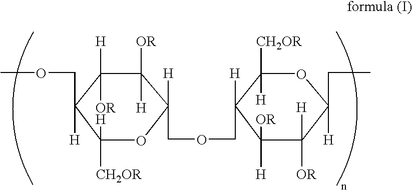
wherein;
R is H, C₁₋₆ alkyl, (C₁₋₆ alkylO)ₘR², or —C(O)R¹, wherein at least one R is H, one R is C₁₋₆ alkyl or (C₁₋₆ alkylO)ₘR², and one R is —C(O)R¹;
R¹ is C₅₋₁₀ aryl or C₁₋₆ alkyl, optionally substituted with at least one CO₂H;
R² is H or C(O)R¹;
m is 1-5; and
n is 2-350.

60. A flexible film comprising;
a tooth whitening agent, a flavorant, and a compound of formula (I); 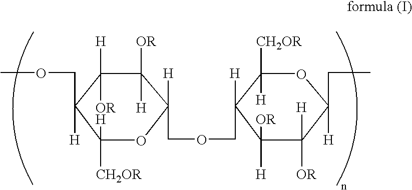
wherein;
R is H, C₁₋₆ alkyl, (C₁₋₆ alkylO)ₘR², or —C(O)R¹, wherein at least one R is H, one R is C₁₋₆ alkyl or (C₁₋₆ alkylO)ₘR², and one R is —C(O)R¹;
R¹ is C₅₋₁₀ aryl or C₁₋₆ alkyl, optionally substituted with at least one CO₂H;
R² is H or C(O)R¹;
m is 1-5; and
n is 2-350.
